(19) United States
(12) Patent Application Publication
(10) Pub. No.: US 2024/0169198 A1
(43) Pub. Date: May 23, 2024

(54) APPARATUS FOR TRAINING DEEP LEARNING MODEL

(71) Applicant: ELECTRONICS AND TELECOMMUNICATIONS RESEARCH INSTITUTE, Daejeon (KR)

(72) Inventors: MUHAMMAD ZUBAIR, Daejeon (KR); Sung Pil WOO, Daejeon (KR); Chang Woo YOON, Daejeon (KR); Sun Hwan LIM, Daejeon (KR)

(21) Appl. No.: 18/242,725

(22) Filed: Sep. 6, 2023

(30) Foreign Application Priority Data
Nov. 17, 2022 (KR) 10-2022-0154663

(51) Int. Cl. G06N 3/08; G06N 3/0442; G06N 3/0464

(52) U.S. Cl. CPC G06N 3/08; G06N 3/0442; G06N 3/0464

(57) ABSTRACT

An apparatus for training a deep learning model for classifying emotions from biosignals includes: a memory configured to store a program for training the deep learning model; and a processor configured to train the deep learning model by executing the program, wherein, when the processor executes the program, the processor inputs an input matrix to an attention layer constituting the deep learning model, the input matrix being composed of a plurality of features each mapped to a plurality of channels and a plurality of feature groups as the biosignals are acquired from a plurality of channels and the biosignals acquired from each channel are divided into the plurality of feature groups, and the attention layer operates to mask the input matrix using an attention matrix in which an importance of features in each channel is reflected.

### CROSS-REFERENCE TO RELATED APPLICATION

This application claims priority to and the benefit of Korean Patent Application No. 10-2022-0154663, filed on Nov. 17, 2022, the disclosure of which is incorporated herein by reference in its entirety.

### BACKGROUND

### 1. Field of the Invention

The present invention relates to an apparatus for training a deep learning model, and more particularly, to an apparatus for training a deep learning model for classifying human emotions.

### 2. Description of Related Art

Emotion computing (or affective computing) is a new field of research and development of artificial intelligence related to designing systems and devices capable of recognizing, interpreting, and processing human emotions, and may be defined as computing that is related to, arises from, or affects emotions. Emotion computing is a multidisciplinary field that develops automatic emotion recognition systems and emotion detection interfaces by integrating knowledge from artificial intelligence, cognitive science, and psychology, and aims to implant human-like emotion recognition and interpretation capabilities into computers by developing robust computational models for recognizing human emotions. Emotional computing systems may be applied to various industries such as mental health monitoring, safe driving, gaming, and security.

Recently, emotion recognition intelligent systems have been used in various fields such as e-health, e-learning, recommender systems, smart homes, smart cities, and intelligent conversation systems. The use of computer-based automatic emotion recognition has great potential in a variety of intelligent systems, including online gaming, neuro-marketing (evaluating customer feedback), and mental health monitoring. For example, a medical system equipped with an emotion recognition module may monitor a patient's mental and physical condition in real time and prescribe appropriate treatment accordingly. The goal of emotion recognition and detection in the field of human computer interaction (HCI) is to design and implement an intelligent system with optimized HCI that may adapt to a user's emotional state.

Meanwhile, biosignals are related to various physiological processes of humans. Biosignals include electroencephalography (EEG), electrocardiography (ECG), electromyography (EMG), galvanic skin response (GSR), body temperature and respiration (RSP), and the like. A biosignal may efficiently capture emotion-related information generated in response to external or internal stimuli. Changes in these signals associated with various emotions are induced by activity of the autonomic nervous system, which controls various body functions such as heart rate, temperature, pupil response, and digestion. One of the major advantages of these biosignals over facial/voice expressions is that they are involuntary and may only be modulated through autonomic activation. For example, sympathetic and parasympathetic components of the autonomic nervous system may regulate heart rate when activated by internal or external stimuli, and similarly, the GSR, EMG, and RSP signals are regulated as a result of the sympathetic and parasympathetic activation.

The emotion recognition systems proposed in most researches for effectively recognizing emotions have limitations in their generalization in that they depend on a specific set of features artificially created by designers. Specifically, morphological characteristics of biosignals vary from person to person depending on the individual's physiological process, mental state, and time flow. Therefore, for the generalization and high performance of an emotion classification model, it is necessary to eliminate the dependence on “hand crafted features” by designers. In addition, a system designed to operate on a specific type of stimulus data may not perform emotion classification effectively on data coming from other stimuli. Physiological patterns for similar stimuli may vary greatly from person to person and from situation to situation, so it is necessary to design a system to obtain ground truth of accurate emotions in consideration of the varying physiological patterns.

When training a deep learning model for emotion classification, “class imbalanced data” in which training data has a skewed distribution of class instances is also a factor causing degradation in the classification performance of the emotion classification deep learning model. In imbalanced data, minority classes are sparsely represented, while another class is abundant, and therefore high representation of the majority class in the training process causes a bias of a classification algorithm toward the majority class, resulting in poor classification performance for minority class samples. However, in real world scenarios, infrequent events are very important. For example, in surveillance tasks, suspicious activity is a rare event that needs to be recognized correctly, and in medical applications, a disease to be diagnosed is also a rare event that needs to be recognized correctly. Therefore, it is necessary to develop a method of training a new model to deal with the model bias due to the imbalanced data distribution.

Numerous standard stimulus databases and derivation approaches have been proposed for inducing various types of emotions, but emotions derived using various types of stimuli in a laboratory environment are far from emotions experienced by humans in everyday life. Therefore, stable and noise-free data generated under controlled conditions in a laboratory environment may not meet the real requirements of emotion recognition. In addition, the standard stimulus database as described above is inefficient for training deep learning architectures due to the limited number of samples. Therefore, it is necessary to minimize data required for training a deep learning-based emotion classification model.

### SUMMARY OF THE INVENTION

An embodiment of the present invention is directed to providing an apparatus for training a deep learning model for improving classification performance of a deep learning model for emotion classification.

According to an exemplary embodiment, an apparatus for training a deep learning model for classifying emotions from biosignals includes: a memory configured to store a program for training the deep learning model; and a processor configured to train the deep learning model by executing the program, in which, when the processor executes the program, the processor inputs an input matrix to an attention layer constituting the deep learning model, the input matrix being composed of a plurality of features each mapped to a plurality of channels and a plurality of feature groups as the biosignals are acquired from a plurality of channels and the biosignals acquired from each channel are divided into the plurality of feature groups, and the attention layer operates to mask the input matrix using an attention matrix in which an importance of features in each channel is reflected.

The attention layer may determine the attention matrix based on channel-wise statistics of an average of the features belonging to each channel and feature-wise statistics of an average of the features belonging to each feature group.

The attention layer may determine total statistics representing an importance of an individual feature in each channel by multiplying the average matrix for each channel and the average matrix for each feature, determine the attention matrix by applying a predefined activation function to the determined total statistics, and multiply the determined attention matrix by the input matrix to mask the input matrix.

The processor may train the deep learning model using an inter-class loss function defining a margin between different classes.

The inter-class loss function may be defined based on (i) a Gaussian similarity that normalizes a margin between a feature and a predicted class of the corresponding feature, and (ii) a hard negative sample defined as a sample that does not belong to a specific class but is classified into the specific class according to the Gaussian similarity.

The inter-class loss function may be defined using (i) a hyperparameter for applying a margin between two classes, (ii) a Gaussian similarity for a target class of a specific hard negative sample, and (iii) a Gaussian similarity for another class of the specific hard negative sample as factors, and the specific hard negative sample may be a sample that belongs to the target class and has a maximum Gaussian similarity for the other class.

The processor may train the deep learning model so that a margin between two classes is extended using the inter-class loss function.

The processor may train the deep learning model using an intra-class loss function defining a margin between samples within the same class.

The intra-class loss function may be defined based on (i) a Gaussian similarity that normalizes a margin between a feature and a predicted class of the corresponding feature, and (ii) a hard positive sample defined as a sample that belongs to the specific class and has a minimum Gaussian similarity for the specific class.

The intra-class loss function may be defined using a Gaussian similarity to a target class of a specific hard positive sample and a Gaussian similarity to a target class of an anchor sample as factors, and the specific hard positive sample may be a sample belonging to the target class, and the anchor sample may be a sample having a maximum Gaussian similarity for the target class.

The processor may train the deep learning model so that variance between samples within a class is reduced using the intra-class loss function.

The processor may train the deep learning model using a final loss function determined by a weighted sum method for an inter-class loss function defining a margin between different classes and an intra-class loss function defining a margin between samples within the same class.

The deep learning model may include one or more convolutional neural network (CNN) layers, one or more long short-term memory (LSTM) layers, and one or more fully connected layers, and the attention layer may be located at a front end of the CNN layer or between the CNN layer and the LSTM layer based on a training data propagation direction in the deep learning model.

The biosignal may be an electroencephalography (EEG) signal or an electrocardiography (ECG) signal.

According to another exemplary embodiment, an apparatus for training a deep learning model includes: a memory configured to store a program for training a multimodal deep learning model including a first deep learning model for classifying emotion from a first biosignal and a second deep learning model for classifying emotion from a second biosignal; and a processor configured to train the deep learning model by executing the program, in which, when the processor executes the program, the first deep learning model inputs a first input matrix acquired from the first biosignal, and the second deep learning model inputs a second input matrix acquired from the second biosignal, and the multi-mode deep learning model is trained to classify emotions from the first and second biosignals through a structure in which outputs of each of the first and second deep learning models are combined in parallel in a fully connected layer.

The first and second input matrices may be composed of a plurality of features each mapped to a plurality of channels and a plurality of feature groups as the first and second biosignals are each acquired from a plurality of channels and the biosignals acquired from each channel are divided into the plurality of feature groups, and the first and second deep learning models may each include an attention layer that operates to mask the input matrix using an attention matrix in which an importance of features in each channel is reflected.

The first and second deep learning models may each include one or more convolutional neural network (CNN) layers and one or more long short-term memory (LSTM) layers, in the first deep learning model, the attention layer may be located at a front end of the CNN layer based on a training data propagation direction in the first deep learning model, and in the second deep learning model, the attention layer may be located between the CNN layer and the LSTM layer based on the training data propagation direction in the second deep learning model.

The first biosignal may be an electroencephalography (EEG) signal and the second biosignal is an electrocardiography (ECG) signal.

### BRIEF DESCRIPTION OF THE DRAWINGS

FIG. 1 is an exemplary view illustrating a methodology for classifying emotions through a deep learning model trained through an apparatus for training a deep learning model according to an embodiment of the present invention.

FIG. 2 is a block diagram illustrating an apparatus for training a deep learning model according to an embodiment of the present invention.

FIG. 3 is an exemplary diagram illustrating architecture of an attention layer in an apparatus for training a deep learning model according to an embodiment of the present invention.

FIG. 4 is an exemplary view illustrating a function of an inter-class loss function in an apparatus for training a deep learning model according to an embodiment of the present invention.

FIG. 5 is an exemplary view illustrating a function of an intra-class loss function in an apparatus for training a deep learning model according to an embodiment of the present invention.

FIGS. 6 and 7 are exemplary views illustrating an implementation example of a network of a deep learning model in the apparatus for training a deep learning model of the present embodiment.

FIG. 8 is a view illustrating an implementation example of a network of a multimodal deep learning model in an apparatus for training a deep learning model according to an embodiment of the present invention.

FIG. 9 is an exemplary view illustrating a zero padding scheme for a heart rate variability (HRV) signal in an apparatus for training a deep learning model according to an embodiment of the present invention.

### DETAILED DESCRIPTION OF EXEMPLARY EMBODIMENTS

Hereinafter, an embodiment of an apparatus for training a deep learning model according to the present invention will be described with reference to the accompanying drawings. In this process, thicknesses of lines, sizes of components, and the like illustrated in the accompanying drawings may be exaggerated for clearness of explanation and convenience. In addition, terms to be described below are defined in consideration of functions in the present disclosure and may be construed in different ways according to the intention of users or practice. Therefore, these terms should be defined on the basis of the content throughout the present specification.

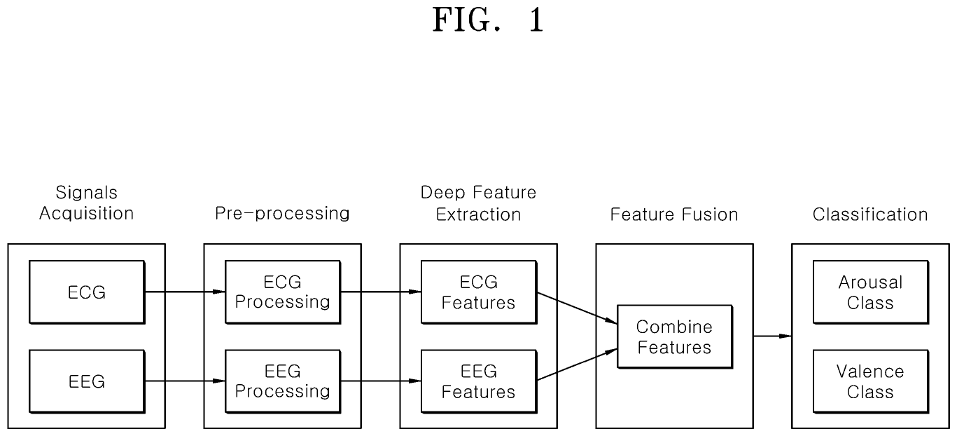

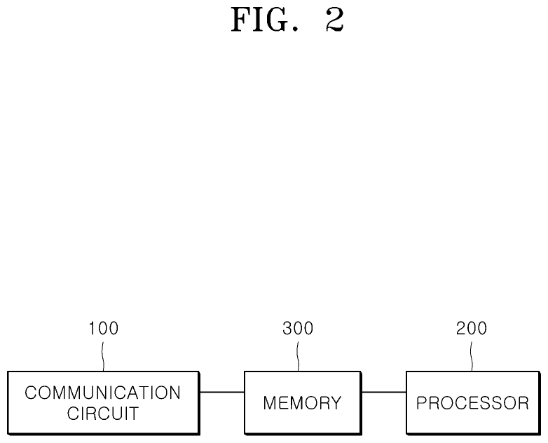

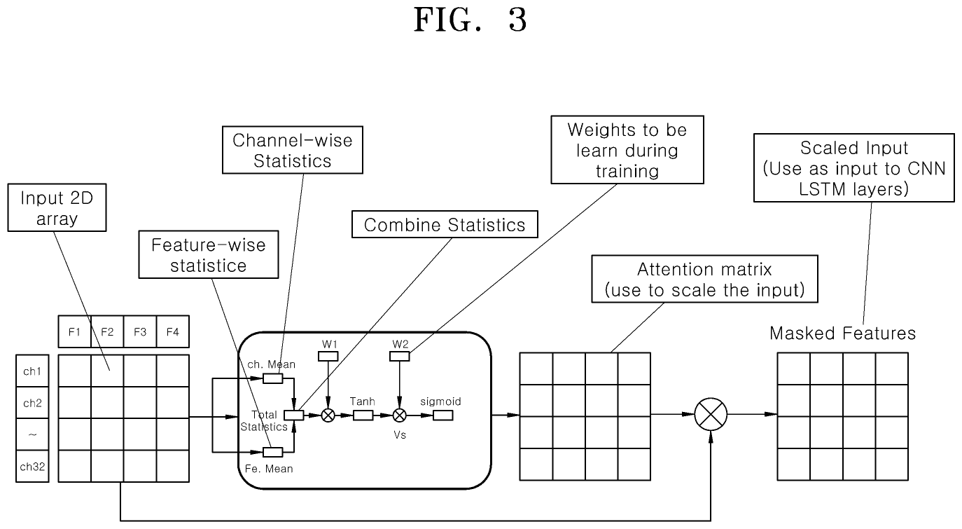

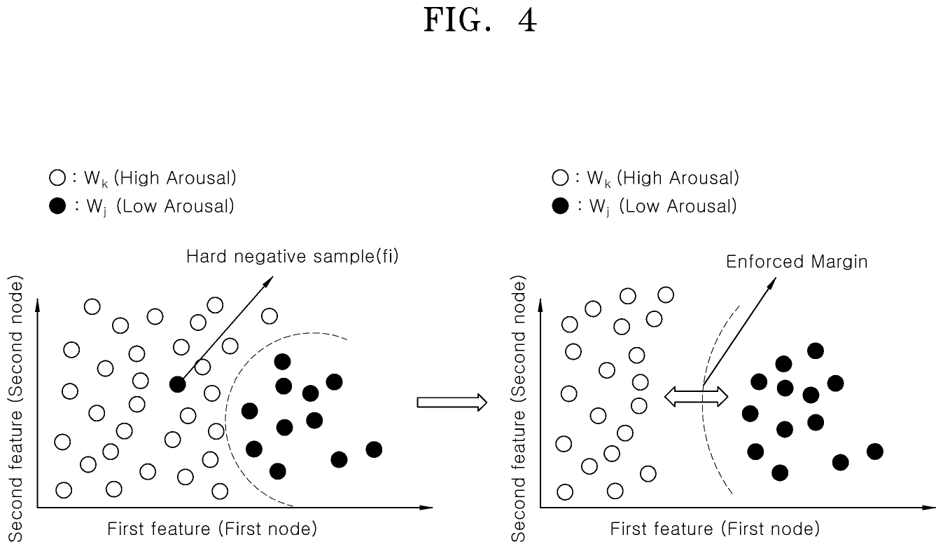

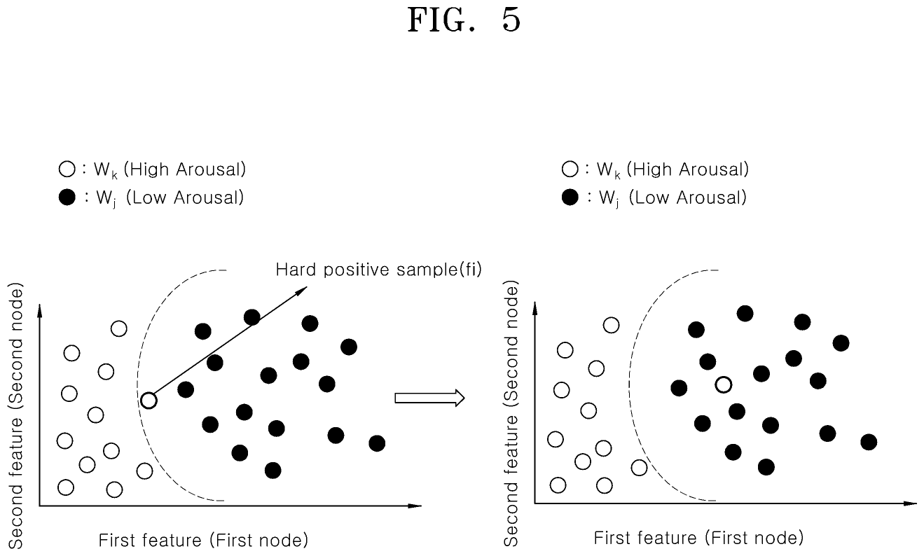

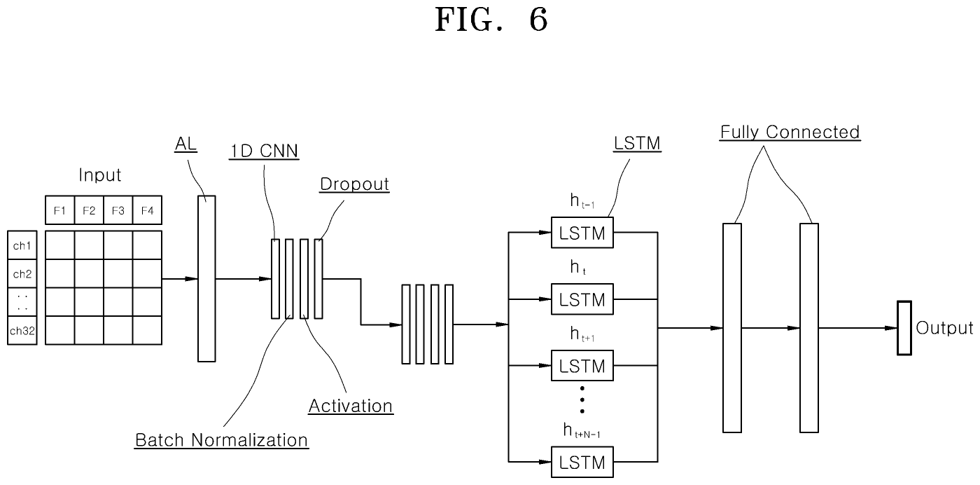

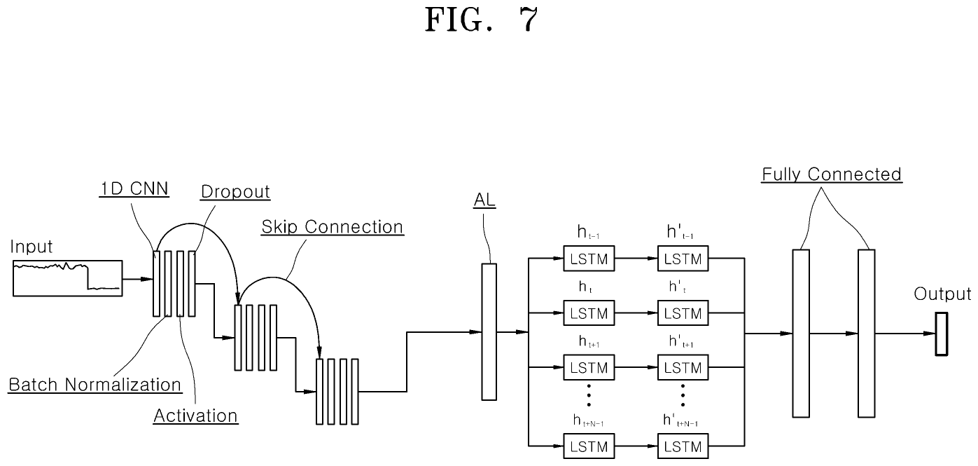

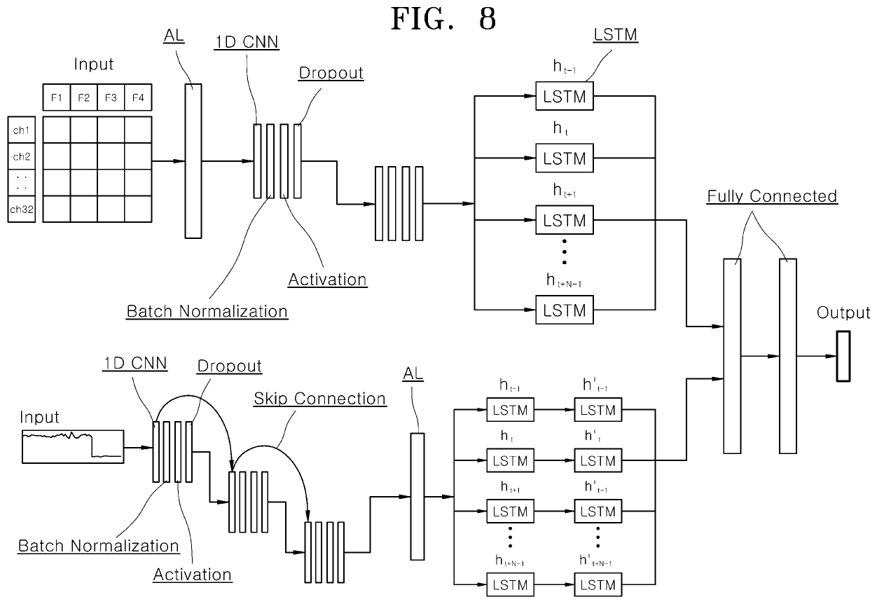

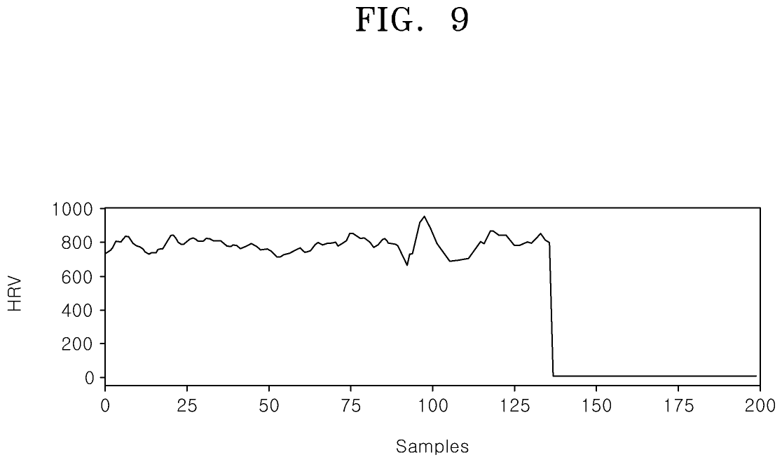

FIG. 1 is an exemplary view illustrating a methodology for classifying emotions through a deep learning model trained through an apparatus for training a deep learning model according to an embodiment of the present invention, FIG. 2 is a block diagram illustrating an apparatus for training a deep learning model according to an embodiment of the present invention, FIG. 3 is an exemplary diagram illustrating architecture of an attention layer in an apparatus for training a deep learning model according to an embodiment of the present invention, FIG. 4 is an exemplary view illustrating a function of an inter-class loss function in an apparatus for training a deep learning model according to an embodiment of the present invention, FIG. 5 is an exemplary view illustrating a function of an intra-class loss function in an apparatus for training a deep learning model according to an embodiment of the present invention, FIGS. 6 and 7 are exemplary views illustrating an implementation example of a network of a deep learning model in the apparatus for training a deep learning model of the present embodiment, FIG. 8 is a view illustrating an implementation example of a network of a multimodal deep learning model in an apparatus for training a deep learning model according to an embodiment of the present invention, and FIG. 9 is an exemplary view illustrating a zero padding scheme for a heart rate variability (HRV) signal in an apparatus for training a deep learning model according to an embodiment of the present invention.

As illustrated in FIG. 1, the present embodiment presents a multimodal emotion classification deep model using a biosignal. The proposed deep model aims to train efficient deep expression of a biosignal by extracting emotion-related information from the biosignal, and improve effective classification performance of neighboring emotions by processing a problem of imbalanced class data, applying a maximum margin (maximum distance) between class boundaries, and reducing variance between samples within a class. Referring to FIG. 1, the emotion classification procedure includes signal acquisition, pre-processing, deep feature extraction, feature fusion, and classification. The present embodiment focuses on the deep feature extraction and classification (design and training of a deep learning model).

1. Configuration for Training Deep Learning Model

Referring to FIG. 2, an apparatus for training a deep learning model of the present embodiment includes a communication circuit 100, a memory 200, and a processor 300.

The communication circuit 100 receives training data (a biosignal) for training the deep learning model from the outside or from a sensor (e.g., an EEG sensor or an ECG sensor) mounted inside the apparatus. The communication circuit 100 may be implemented as wired communication circuits such as a power line communication device, a telephone line communication device, cable home (MoCA), Ethernet, IEEE1294, an integrated wired home network, and an RS-485 control device, or may be implemented as wireless communication circuits such as a wireless LAN (WLAN), Bluetooth, HDR WPAN, UWB, ZigBee, Impulse Radio, 60 GHz WPAN, Binary-CDMA, wireless USB technology, and wireless HDMI.

In the present embodiment, the biosignal may be an electroencephalography (EEG) signal or an electrocardiography (ECG) signal acquired from a plurality of channels (e.g., a plurality of sensor electrodes), and it is premised that the biosignals acquired from each channel are divided into a plurality of feature groups. In the case of the EEG signal, a plurality of feature groups may be defined according to an EEG frequency band (e.g., θ (4 to 7 Hz), α (8 to 13 Hz), β (14 to 30 Hz), and γ (31 to 50 Hz)), and in the case of the ECG signal, a plurality of feature groups may be defined according to an R-Peak value.

The memory 200 stores the biosignal received through the communication circuit 100 and a program for training a deep learning model. The memory 200 may be implemented as NAND flash memories such as a compact flash (CF) card, a secure digital (SD) card, a memory stick, a solid-state drive (SSD), and a micro SD card, magnetic computer storage devices such as a hard disk drive (HDD), and optical disc drives such as CD-ROM and DVD-ROM drives, and the like.

The processor 300 corresponds to a subject that trains the deep learning model by executing the program stored in the memory 200. The processor 300 may be implemented as a central processing unit (CPU) or a system on chip (SoC), and may operate an operating system or applications to control a plurality of hardware or software components connected to the processor 300, thereby performing various data processing and operations. The processor 300 may be configured to execute at least one command stored in the memory 200 and store the execution result data in the memory 200.

The processor 300 generates an input matrix input to the deep learning model through pre-processing of the biosignal stored in the memory 200. As described above, the biosignals are acquired from a plurality of channels, and the biosignals acquired from each channel are divided into a plurality of feature groups. Based on this, the processor 300 generates an input matrix composed of a plurality of features (feature vectors) each mapped to a plurality of channels and a plurality of feature groups from the biosignals. The input matrix is input to the attention layer (described later) of the deep learning model and used as training data for learning the deep learning model.

2. Attention Layer

The principle of the attention mechanism in the deep learning is to focus only on task-related information, and expand the input according to the importance of the information. Therefore, the highest relevant information is propagated through the deep learning model, and classification performance is improved because the information is suitable for classification due to its high correlation with the target task. In addition, the exclusion of irrelevant information from the attention mechanism improves classification performance by preventing feature abstraction due to contamination with irrelevant features.

In the present embodiment, focusing on the fact that each channel from which biosignals are acquired and each feature belonging to each channel have different contributions to emotion classification, an attention layer that operates to mask an input matrix using each channel and an attention matrix in which the importance of features in each channel is reflected is employed.

FIG. 3 illustrates the architecture of the attention layer. The attention layer determines, from the input matrix, channel-wise statistics of an average of features belonging to each channel and feature-wise statistics of an average of features belonging to each feature group, and determines total statistics representing the importance of individual features in each channel by multiplying the channel-wise statistics and the feature-wise statistics. A predefined activation function is applied to the determined total statistics to determine the attention matrix. Finally, the attention layer masks the input matrix by multiplying the attention matrix by the input matrix. Hyperbolic tangent (Tan h) and sigmoid (Sigmoid) may be applied as the above activation function. Equation 1 below shows a process in which the input matrix is masked by calculating the attention matrix.

$${Channel}_{mean} = \frac{1}{c} {\sum }_{n = 1}^{c} {f}_{n , k} \qquad [ Equation 1 ]$$

$${Feature}_{mean} = \frac{1}{k} {\sum }_{n = 1}^{k} {f}_{c , n}$$

$${Total}_{stat} = {Channel}_{mean} * {Feature}_{mean}$$

$${V}_{s} = tanh ( {Total}_{stat} * W 1 + b 1 )$$

$${A}_{c , k} = sigmoid ( {V}_{s} * W 2 + b 2 )$$

$${F}_{masked} = {F}_{c , k} * {A}_{c , k}$$

In Equation 1, f_{c,k} denotes a feature vector corresponding to channel c and feature group k in an input matrix F′_{c,k}, and Channelₘₑₐₙ, Featureₘₑₐₙ, and Totalₛₜₐₜ denote the channel-wise statistics, the feature-wise statistics, and the total statistics, respectively. W1, W2, b1, and b2 denote training parameters that are optimized in the process of training the deep learning model. A_{c,k} denotes the attention matrix and F_{masked} denotes the masked input matrix.

### 3. Loss Function

### 3-1. Gaussian Similarity and Hard Sample

The loss function employed in the present embodiment performs a function of preventing model bias of majority classes caused by class imbalanced data and ensuring effective classification of neighboring emotions. To this end, the loss function is defined to i) extend margins between different classes and ii) reduce variance between samples within a class. The best way to achieve the above i) and ii) is to measure class similarity using feature space representation instead of class prediction. In this way, a class cluster compression may be induced at a feature level. Based on the above viewpoint, in the present embodiment, instead of using Euclidean similarity at a class level, the Gaussian similarity is used at an instance level (sample level). The Gaussian similarity (d(fᵢ, wⱼ)) calculated by Bergman divergence is as shown in Equation 2 below.

$$d ( {f}_{i} , {w}_{j} ) = exp ( - \frac{{‖ {f}_{i} - {w}_{j} ‖}^{2}}{σ} ) \qquad [ Equation 2 ]$$

In Equation 2, σ is a weight parameter that normalizes a margin between a feature fᵢ and a predicted class wⱼ of the feature fᵢ. The Gaussian similarity according to Equation 2 provides flexibility with which samples can be processed at the feature expression level, thereby inducing compression of class clusters according to margins between boundaries of different classes and variance reduction among samples within a class.

Before defining the loss function, a hard sample is defined based on the Gaussian similarity. A hard positive sample is defined as a sample that belongs to a specific class “c” (i.e., its label is class “c”) and has a minimum Gaussian similarity for the specific class “c.” That is, a sample far from the center of the specific class “c” corresponds to the hard positive sample. The hard negative sample is defined as a sample that does not belong to the specific class “c” (i.e., its label is not class “c”), but is classified into the specific class “c” according to the Gaussian similarity according to Equation 2. That is, a sample that belongs to another class “d” but is located near the specific class “c” in a feature space corresponds to a hard negative sample. The hard positive sample Pᵢ^{ins} and the hard negative sample Nᵢ^{ins} may be defined according to Equation 3 below.

pᵢ^{ins}={xᵢ|αᵢ=c,less similarity d(fᵢ,wⱼ)}

Nᵢ^{ins}={xᵢ|αᵢ≠c,High similarity d(fᵢ,wⱼ)} [Equation 3]

### 3-2. Inter-Class Loss Function

The inter-class loss function specifies a margin between different classes and is applied to maximize a margin between boundaries of different classes. By applying the maximum margin between the boundaries of different class, misclassification of neighboring emotions and misclassification of samples from minority classes may be eliminated.

The inter-class loss function is defined based on the above-described Gaussian similarity and hard negative sample, and specifically, is defined using (i) a hyperparameter for applying a margin between two classes, (ii) a Gaussian similarity for a target class of a specific hard negative sample, and (iii) a Gaussian similarity for another class of the specific hard negative sample as factors. Here, the specific hard negative sample is a sample that belongs to the target class and has a maximum Gaussian similarity for another class.

When the specific hard negative sample is the target class is wⱼ, the other class is wₖ, and the hyperparameter is β, the inter-class loss function L^{mm} is defined as in Equation 4 below.

L^{mm}=∥β−d(fᵢ,wⱼ)−max{d(fᵢ,wₖ)}∥² [Equation 4]

Referring to FIG. 4, the specific hard negative sample fᵢ belongs to a target class wⱼ and has a maximum Gaussian similarity for the other class wₖ. The margin between the classes wⱼ and wₖ is maximized through the inter-class loss function according to Equation 4, thereby reducing the possibility of the sample fᵢ being misclassified as the class wₖ.

### 3-3. Intra-Class Loss Function

The intra-class loss function specifies a margin between samples within the same class and is applied to minimize variance between samples within the class.

The intra-class loss function is defined based on the above-described Gaussian similarity and hard positive sample, and is specifically defined using (i) a Gaussian similarity for a target class of a specific hard positive sample and (ii) a Gaussian similarity for a target class of an anchor sample as factors. Here, the specific hard positive sample corresponds to a sample that belongs to the target class, and the anchor sample corresponds to a sample that has a maximum Gaussian similarity for the target class.

When the specific hard positive sample is fᵢ, the anchor sample is fₖ, and the target class is wᵢ, the intra-class loss function L^{mv} is defined as in Equation 5 below.

L^{mv}=∥d(fₖ,wⱼ)−min{d(f_{d},wⱼ)}∥² [Equation 5]

In Equation 5, d(fₖ, wⱼ) denotes the maximum Gaussian similarity for the anchor sample, and min{d(fᵢ, wⱼ)} denotes the minimum Gaussian similarity for the specific hard positive sample.

Referring to FIG. 5, the specific hard positive sample fᵢ belongs to the target class wⱼ and has the minimum Gaussian similarity. The variance between samples in the class wⱼ is reduced through the intra-class loss function according to Equation (i.e., the class cluster is compressed), reducing the possibility of the sample fᵢ being misclassified as the class wₖ.

### 3-4. Final Loss Function

The final loss function is determined by a weighted sum method for the inter-class loss function and the intra-class loss function, and follows Equation 6 below.

L^{mvmm}=α*L^{mw}+β*L^{mm} [Equation 6]

In Equation 6, α and β are weights for the inter-class loss function and the intra-class loss function, respectively, and may be defined based on the designer's experimental results (e.g., α=β=0.5).

Since the final loss function uses both the hard positive and hard negative samples, it functions to maximize the margin between different classes and minimize the variance between samples within a class.

### 4. Deep Learning Model Network Architecture

The single-mode deep learning model may be configured to include one or more convolutional neural network (CNN) layers, one or more long short-term memory (LSTM) layers, and one or more fully connected layers, and the location of the above-described attention layer may be determined according to the type of biosignal. Hereinafter, the deep learning model network architecture will be described by being divided into EEG-based emotion classification, ECG-based emotion classification, and multimodal classification.

### 4-1. EEG-Based Classification (Single Mode)

Referring to FIG. 6, in the present embodiment, the deep learning model for EEG-based emotion classification includes two CNN layers, one LSTM layer, and two fully connected layers. Each CNN layer may be used with a batch normalization layer, an activation layer (e.g., parametric ReLu), and a dropout layer.

32×4 input tensors based on “channel×characteristic group” were used as an input matrix, and mutual relationships between different channels may be learned. When 4×32 input tensors are used as an input matrix, a mutual relationship between the features of the EEG frequency band may be learned.

The location of the attention layer AL may be determined so that a suitable channel and characteristic group (EEG frequency band) is selected for emotion classification. In the present embodiment, the attention layer AL is located at a front end of the CNN layer based on a training data propagation direction. The attention layer AL operates to mask the input matrix by assigning trained weights to each channel and features of the channels. The attention matrix is used to assign weights to an input and thus information with high importance is propagated in the deep learning model, and effective training may be achieved.

### 4-2. EEG-Based Classification (Single Mode)

Referring to FIG. 7, in the present embodiment, the deep learning model for the EEG-based emotion classification includes three CNN layers, two LSTM layer, and two fully connected layers. Each CNN layer may be used with a batch normalization layer, an activation layer (e.g., parametric ReLu), and a dropout layer. To provide attention to spatial features extracted from a heart rate variability (HRV) signal, the location of the attention layer AL is selected between the CNN layer and the LSTM layer.

### 4-3. Multimodal Classification

As illustrated in FIG. 8, the multimodal deep learning model has a structure in which outputs of a deep learning model for EEG-based emotion classification and a deep learning model for ECG-based emotion classification are combined in parallel in the fully connected layer, and thus is trained to classify emotions from the EEG signal and the ECG signal.

For an EEG branch in the multimodal deep learning model, a CNN kernel, a CNN feature map, batch normalization, a dropout ratio, a kernel normalization ratio, and a kernel initialization and activation function may be selected from results trained in the single mode, and the number of units in the LSTM layer combined with a kernel initializer, a recurrent regularizer, and a recurrent dropout rate may remain the same as in the single mode. In the case of the ECG branch, the same structure as that of the single mode may also be applied.

### 5. Classification Performance Verification

### 5-1. Input Data

As the pre-processing of the EEG signal, features of each EEG frequency band were calculated through differential entropy (DE). First, the EEG signals of all channels were divided into frequency bands of θ, α, β, and γ, and the differential entropy was calculated for each frequency band of all channels according to Equation 7 below.

$$f ( X ) = {\int }_{- ∞}^{∞} \frac{1}{\sqrt{2 {πσ}^{2}}} exp \frac{{( x - μ )}^{2}}{2 {πσ}^{2}} log \frac{1}{\sqrt{2 {πσ}^{2}}} \qquad [ Equation 7 ]$$

$$exp \frac{{( x - μ )}^{2}}{2 {πσ}^{2}} = \frac{1}{2} log 2 π e {σ}^{2}$$

In the case of the ECG signal, the HRV series was calculated after R-peak was detected, and the ECG signal was normalized before the HRV series. To set segments of the same length, as illustrated in FIG. 9, a zero-padding scheme that adds 0 to ends of each sample is applied.

### 5-2. EEG Signal-Based Emotion Classification Result

For the EEG signal-based emotion classification, a database for emotion analysis using physiological signals (DEAP) dataset was used. After the EEG signals of 32 channels were first divided into frequency bands of θ, α, β, and γ, the DE features were calculated, and 32×4 input tensors were applied to the model training.

Three scenarios were applied in the EEG signal-based emotion classification experiment.

\textcircled{1} entropy loss function without the attention layer. In this case, accuracy of 68.34% for an “arousal” class and 67.68% for a “valence” class were achieved.

\textcircled{2} Next, the deep learning model was tested by arranging the attention layer and applying the cross-entropy loss function. In this case, accuracy of 90.23% for the “arousal” class and 89.36% for the “valence” class were achieved. Through this test, the effect of the attention layer was demonstrated.

\textcircled{3} Next, the deep learning model was tested by arranging the attention layer and applying the loss function according to Equations 4 to 6 above. In this case, accuracy of 94.26% for the “arousal” class and 93.40% for the “valence” class were achieved. Through this test, the effect of the loss function presented in the present embodiment was verified.

Table 1 below shows the EEG signal-based emotion classification test results.

TABLE 1

| Classes | Arousal | Valence |
|---|---|---|
| CNN/LSTM + | 68.34% | 67.68% |
| cross-entropy | | |
| Attention layer + | 90.23% | 89.36% |
| CNN/LSTM + cross- | | |
| entropy | | |
| Attention layer + | 94.26% | 93.40% |
| CNN/LSTM + proposed | | |
| loss function | | |

### 5-3. ECG Signal-Based Emotion Classification Result

For the ECG signal-based emotion classification, ECG data from the AMIGOS database was applied.

Three scenarios were applied in the ECG signal-based emotion classification experiment.

\textcircled{1} entropy loss function without the attention layer. In this case, accuracy of 69% for the “arousal” class and 71% for the “valence” class were achieved.

\textcircled{2} Next, the deep learning model was tested by arranging the attention layer and applying the cross-entropy loss function. In this case, accuracy of 72.45% for the “arousal” class and 77.7% for the “valence” class were achieved. Through this test, the effect of the attention layer was demonstrated.

\textcircled{3} Next, the deep learning model was tested by arranging the attention layer and applying the loss function according to Equations 4 to 6 above. In this case, accuracy of 78.55% for the “arousal” class and 82.35% for the “valence” class were achieved. Through this test, the effect of the loss function presented in the present embodiment was verified.

Table 2 below shows the ECG signal-based emotion classification test results.

TABLE 2

| Classes | Arousal | Valence |
|---|---|---|
| CNN/LSTM + | 69% | 71% |
| cross-entropy | | |
| Attention layer + | 72.45% | 77.7% |
| CNN/LSTM + cross- | | |
| entropy | | |
| Attention layer + | 78.55% | 82.35% |
| CNN/LSTM + proposed | | |
| loss function | | |

### 5-4. Multimodal Emotion Classification Result

Three scenarios were applied in the multimodal emotion classification experiment.

\textcircled{1} entropy loss function without the attention layer. In this case, accuracy of 73.45% for the “arousal” class and 68.44% for the “valence” class were achieved.

\textcircled{2} Next, the deep learning model was tested by arranging the attention layer and applying the cross-entropy loss function. In this case, accuracy of 87.3% for the “arousal” class and 87.72% for the “valence” class were achieved. Through this test, the effect of the attention layer was demonstrated.

\textcircled{3} Next, the deep learning model was tested by arranging the attention layer and applying the loss function according to Equations 4 to 6 above. In this case, accuracy of 90.54% for the “arousal” class and 89.16% for the “valence” class were achieved. Through this test, the effect of the loss function presented in the present embodiment was verified.

Table 2 below shows the ECG signal-based emotion classification test results.

TABLE 3

| Classes | Arousal | Valence |
|---|---|---|
| CNN/LSTM + | 73.45% | 68.44% |
| cross-entropy | | |
| Attention layer + | 87.3% | 87.72% |
| CNN/LSTM + cross- | | |
| entropy | | |
| Attention layer + | 90.54% | 89.16% |
| CNN/LSTM + proposed | | |
| loss function | | |

As described above, according to the present invention, i) through an attention layer that operates to mask an input matrix using an attention matrix reflecting the importance of features as well as channels, by ensuring that only important features are propagated and excluding irrelevant features that cause degradation of classification performance, it is possible to improve emotion classification performance of the deep learning model, and ii) by training the deep learning model by applying an inter-class loss function to expand a margin between classes and an intra-class loss function to reduce variance between samples within a class, it is possible to prevent model bias of majority classes caused by class imbalanced data and to effectively classify neighboring emotions.

Implementations described herein may be implemented in, for example, a method or process, an apparatus, a software program, a data stream, or a signal. Although discussed only in the context of a single form of implementation (e.g., discussed only as a method), implementations of the discussed features may also be implemented in other forms (e.g., an apparatus or a program). The apparatus may be implemented in suitable hardware, software, firmware, and the like. A method may be implemented in an apparatus such as a processor, which is generally a computer, a microprocessor, an integrated circuit, a processing device including a programmable logic device, or the like. Examples of the processor also include communication devices such as a computer, a cell phone, portable/personal digital assistants (“PDA”), and other devices that facilitate communication of information between end-users.

According to an apparatus for training a deep learning model of the present invention, i) through an attention layer that operates to mask an input matrix using an attention matrix reflecting the importance of features as well as channels, by ensuring that only important features are propagated, excluding irrelevant features that cause degradation of classification performance, it is possible to improve emotion classification performance of the deep learning model, and ii) by training the deep learning model by applying an inter-class loss function to expand a margin between classes and an intra-class loss function to reduce variance between samples within a class, it is possible to prevent model bias of majority classes caused by class imbalanced data and to effectively classify neighboring emotions.

Although the present invention has been described with reference to embodiments shown in the accompanying drawings, it is only an example. It will be understood by those skilled in the art that various modifications and other equivalent exemplary embodiments are possible from the present invention. Accordingly, the true technical scope of the present invention is to be determined by the spirit of the appended claims.

What is claimed is:

1. An apparatus for training a deep learning model for classifying emotions from biosignals, the apparatus comprising:
a memory configured to store a program for training the deep learning model; and
a processor configured to train the deep learning model by executing the program,
wherein, when the processor executes the program, the processor inputs an input matrix to an attention layer constituting the deep learning model, the input matrix being composed of a plurality of features each mapped to a plurality of channels and a plurality of feature groups as the biosignals are acquired from a plurality of channels and the biosignals acquired from each channel are divided into the plurality of feature groups, and
the attention layer operates to mask the input matrix using an attention matrix in which an importance of features in each channel is reflected.

2. The apparatus of claim 1, wherein the attention layer determines the attention matrix based on channel-wise statistics of an average of the features belonging to each channel and feature-wise statistics of an average of the features belonging to each feature group.

3. The apparatus of claim 2, wherein the attention layer determines total statistics representing an importance of an individual feature in each channel by multiplying the average matrix for each channel and the average matrix for each feature, determines the attention matrix by applying a predefined activation function to the determined total statistics, and multiplies the determined attention matrix by the input matrix to mask the input matrix.

4. The apparatus of claim 1, wherein the processor trains the deep learning model using an inter-class loss function defining a margin between different classes.

5. The apparatus of claim 4, wherein the inter-class loss function is defined based on (i) a Gaussian similarity that normalizes a margin between a feature and a predicted class of the corresponding feature, and (ii) a hard negative sample defined as a sample that does not belong to a specific class but is classified into the specific class according to the Gaussian similarity.

6. The apparatus of claim 5, wherein the inter-class loss function is defined using (i) a hyperparameter for applying a margin between two classes, (ii) a Gaussian similarity for a target class of a specific hard negative sample, and (iii) a Gaussian similarity for another class of the specific hard negative sample as factors, and
the specific hard negative sample is a sample that belongs to the target class and has a maximum Gaussian similarity for the other class.

7. The apparatus of claim 6, wherein the processor trains the deep learning model so that a margin between two classes is extended using the inter-class loss function.

8. The apparatus of claim 1, wherein the processor trains the deep learning model using an intra-class loss function defining a margin between samples within the same class.

9. The apparatus of claim 8, wherein the intra-class loss function is defined based on (i) a Gaussian similarity that normalizes a margin between a feature and a predicted class of the corresponding feature, and (ii) a hard positive sample defined as a sample that belongs to the specific class and has a minimum Gaussian similarity for the specific class.

10. The apparatus of claim 9, wherein the intra-class loss function is defined using a Gaussian similarity to a target class of a specific hard positive sample and a Gaussian similarity to a target class of an anchor sample as factors, and
the specific hard positive sample is a sample belonging to the target class, and the anchor sample is a sample having a maximum Gaussian similarity for the target class.

11. The apparatus of claim 10, wherein the processor trains the deep learning model so that variance between samples within a class is reduced using the intra-class loss function.

12. The apparatus of claim 1, wherein the processor trains the deep learning model using a final loss function determined by a weighted sum method for an inter-class loss function defining a margin between different classes and an intra-class loss function defining a margin between samples within the same class.

13. The apparatus of claim 1, wherein the deep learning model includes one or more convolutional neural network (CNN) layers, one or more long short-term memory (LSTM) layers, and one or more fully connected layers, and
the attention layer is located at a front end of the CNN layer or between the CNN layer and the LSTM layer based on a training data propagation direction in the deep learning model.

14. The apparatus of claim 1, wherein the biosignal is an electroencephalography (EEG) signal or an electrocardiography (ECG) signal.

15. An apparatus for training a deep learning model, comprising:
a memory configured to store a program for training a multimodal deep learning model including a first deep learning model for classifying emotion from a first biosignal and a second deep learning model for classifying emotion from a second biosignal; and
a processor configured to train the deep learning model by executing the program,
wherein, when the processor executes the program, the first deep learning model inputs a first input matrix acquired from the first biosignal, and the second deep learning model inputs a second input matrix acquired from the second biosignal, and
the multi-mode deep learning model is trained to classify emotions from the first and second biosignals through a structure in which outputs of each of the first and second deep learning models are combined in parallel in a fully connected layer.

16. The apparatus of claim 15, wherein the first and second input matrices are composed of a plurality of features each mapped to a plurality of channels and a plurality of feature groups as the first and second biosignals are each acquired from a plurality of channels and the biosignals acquired from each channel are divided into the plurality of feature groups, and
the first and second deep learning models each include an attention layer that operates to mask the input matrix using an attention matrix in which an importance of features in each channel is reflected.

17. The apparatus of claim 16, wherein the first and second deep learning models each include one or more convolutional neural network (CNN) layers and one or more long short-term memory (LSTM) layers,
in the first deep learning model, the attention layer is located at a front end of the CNN layer based on a training data propagation direction in the first deep learning model, and
in the second deep learning model, the attention layer is located between the CNN layer and the LSTM layer based on the training data propagation direction in the second deep learning model.

18. The apparatus of claim 17, wherein the first biosignal is an electroencephalography (EEG) signal and the second biosignal is an electrocardiography (ECG) signal.
